(19) United States
(12) Patent Application Publication
(10) Pub. No.: US 2020/0377851 A1
(43) Pub. Date: Dec. 3, 2020

(54) Method for the Cultivation of Glial Cells

(71) Applicant: Jude Nicolas Raia, Birmingham, AL (US)

(72) Inventor: Jude Nicolas Raia, Birmingham, AL (US)

(21) Appl. No.: 16/429,006

(22) Filed: Jun. 2, 2019

(51) Int. Cl. C12N 5/07

(52) U.S. Cl. CPC C12N 5/0601; A61K 35/30

(57) ABSTRACT

The present invention provides a method for the cultivation of glial cells from Aurelia aurita Medusa primary cultures. There are no present methods providing instructions on the cultivation of glial cells from any invertebrates. Through the creation of this new method, the growth of glial cells from Aurelia aurita is now possible and the production of new medicines for the treatment of myelination disorders will be faster and easier leading to these medicines becoming more readily available to the consumers who need them.

### TECHNICAL FIELD

The present invention proposes a method for the cultivation of glial cells from Aurelia aurita Medusa primary cultures.

### BACKGROUND ART

The discovery of glial cells in any invertebrate has never been previously performed and as such, no prior methods have ever been developed.

### SUMMARY OF INVENTION

The object of the present invention is to cultivate glial cells from Aurelia aurita Medusa primary cultures. The method proposed here is as follows:

To make the salt water media to grow the cells in, one must make 500 mL of filter sterilized salt water at 1.022 specific gravity and combine with 5 mL of penicillin, 5 mL of the amino acid called, and 1 mL of Gentamicin.

To make the mammalian cell culture media, one must combine 50 mL of fetal bovine serum (FBS), 5 mL of, 5 mL of penicillin, 1 mL of Gentamicin, and 450 mL of Dulbecco's modified Eagle's medium (DMEM).

To harvest the glial cells, one must force tissue from a Medusa stage Aurelia aurita through a cell sieve using a pestle and the aforementioned salt water media and collect in a 15 mL conical. Then bring the tissue and media mixture to 5 mL using more salt water media. Now pour 1 mL into five, new 15 mL conicals and add 1 mL of the digestive enzyme, trypsin and place in an incubator set at 37° C. for 2 minutes. Next, take the conicals and add 1 mL of the mammalian cell culture media to end the digestive reaction caused by the trypsin. Split the now 3 mL in each conical to 1.5 mL and put the excess in five, new 15 mL conicals to have a total of ten, 15 mL conicals. Add 8.5 mL of the mammalian cell culture media to each of the conicals and one must then spin the conicals in a centrifuge at 1000 rpm for five minutes to cause a small pellet to form in the bottom of each conical. Then, decant the supernatant by pouring to leave behind only the pellet. One must now resuspend the pellet in 3 mL of fresh salt water media and plate each conical in plastic cell culture plates. Store plates in an incubator at 18° C. and give 1 mL of fresh salt water to each plate every other day. After one week, glial cells begin to form in the bottom of each plate.

### INDUSTRIAL APPLICABILITY

The industrial applicability of this invention is that medicines for myelination disorders and diseases, such as Parkinson's, Alzheimer's, and Multiple Sclerosis can be created faster and easier using the glial cells found in invertebrates rather than vertebrates. As a jellyfish's DNA is far less complicated than a human's or rat's, drugs, such as monoclonal antibodies, that repair glial cell demyelination could be produced far faster and easier allowing for the cheaper production of the drug and as a result, it becoming more readily available to consumers.

1. The method described previously that cultivates the growth of glial cells harvested specifically from Aurelia aurita Medusa primary cell cultures is characterized in that this method has never been performed before.

2. The method as claimed in claim 1 is characterized in that the growth of glial cells from invertebrates has never been done before as glial cells were previously thought to have only resided in vertebrate creatures.
